(19) United States
(12) Patent Application Publication
(10) Pub. No.: US 2011/0237824 A1
(43) Pub. Date: Sep. 29, 2011

(54) Process for Producing Fluoroalkylsulfonylaminoethyl Alpha-Substituted-Acrylate

(75) Inventors: Takehisa Ishimaru, Saitama (JP); Ryo Nadano, Saitama (JP); Makoto Matsuura, Saitama (JP)

(73) Assignee: Central Glass Company, Limited, Ube-shi (JP)

(21) Appl. No.: 13/131,487

(22) PCT Filed: Dec. 22, 2009
(86) PCT No.: PCT/JP2009/071294

§ 371 Date: May 26, 2011

(30) Foreign Application Priority Data
Dec. 26, 2008 (JP) 2008-334139
Dec. 8, 2009 (JP) 2009-278548

(51) Int. Cl. C07C 307/02

(52) U.S. Cl. 560/150; 564/96

(57) ABSTRACT

Aminoethanol is reacted with a fluoroalkylsulfonic anhydride to obtain a fluoroalkylsulfonylaminoethanol (first step: sulfonylamidation step). The fluoroalkylsulfonylaminoethanol is esterified with an α-substituted acrylate derivative to obtain a desired fluoroalkylsulfonylaminoethyl α-substituted-acrylate (second step: esterification step). Thus, the desired fluoroalkylsulfonylaminoethyl α-substituted-acrylate having a higher purity can be produced in higher yield with higher operating efficiency than in conventional techniques.

### TECHNICAL FIELD

The present invention relates to a process for producing a fluoroalkylsulfonylaminoethyl α-substituted-acrylate represented by the general formula [5]

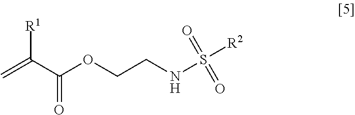

(where; R¹ in the formula represents a hydrogen atom, methyl group, ethyl group, n-propyl group, isopropyl group, n-butyl group, sec-butyl group, tert-butyl group, fluoromethyl group, difluoromethyl group, trifluoromethyl group or perfluoroethyl group; and R² represents a fluoroalkyl group having 1 to 6 carbon atoms.) and serving as a useful compound as a monomer applicable to the next generation photoresist, and to a process for producing a fluoroalkylsulfonylaminoethanol represented by the general formula [3]

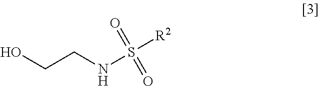

(where R² is the same to that in the general formula [5].) and useful as a raw material therefor.

### BACKGROUND OF THE INVENTION

Sulfonylaminoethyl α-substituted-acrylate is a promising compound as a monomer for use in the next generation photoresist. A resist containing the above-mentioned monomer as its component is known to be excellent in light-transparence ability, surface adhesion and solubility, and to improve development of the resist (Patent Publication 1 and Patent Publication 2).

There is set forth in Patent Publication 3 that a resist composition consisting of a fluorosulfonamide-containing polymer produced by using the above-mentioned monomer is excellent in etching resistance as a resist material and solubility in a developing solution so as to be useful as a negative resist composition.

A fluoroalkylsulfonylaminoethyl α-substituted-acrylate represented by the general formula [5] combines an ester moiety with a sulfonamide moiety, so that a process for producing the same is considered the following two possible production processes, depending on the sequence of reactions.

(a) A process of esterifying hydrochloride of ethanolamine with an α-substituted acrylic acid derivative and then sulfonylamidating the same in the use of a fluoroalkylsulfonic acid derivative

(b) A process of sulfonylamidating ethanolamine with a fluoroalkylsulfonic acid derivative and then esterifying the same in the use of an α-substituted acrylic acid derivative

Concerning the reaction of the process (a), there is disclosed in Patent Publication 4 a method for reacting an aminoethyl α-substituted acrylate or a salt thereof obtained by sulfonylamidation with a fluoroalkylsulfonic halide or a fluoroalkylsulfonic anhydride in a solvent in the presence of a specified base thereby synthesizing the fluoroalkylsulfonylaminoethyl α-substituted-acrylate represented by the general formula [5] (the following scheme). The same process is also disclosed in Examples of Patent Publication 3, in which an example of sulfonic acid-amidating a hydrochloride of a commercially available 2-aminoethylmethacrylate with trifluoromethanesulfonyl chloride.

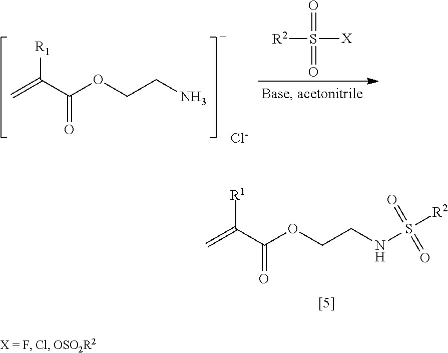

(In the formula, R¹ and R² represent the same to the above.)

On the other hand, concerning the process (b), Patent Publication 1 and Patent Publication 2 set forth only a process for producing an α-substituted-acrylate which process was based on a broad concept of the latter reaction (the esterification reaction), and did no more than disclose a process of synthesizing the same by performing condensation after the slow addition of a methylene chloride solution of a slight excess of α-substituted acrylic acid chloride to a methylene chloride solution of a corresponding alcohol (in this case, the general formula [3]) in the presence of an acid scavenger (generally pyridine or triethylamine), so that there is no description about production of a fluoroalkylsulfonylaminoethanol represented by the general formula [3].

Furthermore, there is known an example where trifluoromethanesulfonic anhydride is reacted with 1-bicyclo[2.2.1]hept-5-en-2-ylmethaneamine in anhydrous methylene chloride in the presence of triethylamine serving as a base, as a process for producing a fluoroalkylsulfonic acid amide derivative (Non-Patent Publication 1). However, a reaction caused in the case of reacting trifluoromethanesulfonic anhydride with a compound such as aminoethanol having both amino group and hydroxyl group and more specifically having two sites reactive with a sulfonating reagent has not been known.

### REFERENCES ABOUT PRIOR ART

### Patent Publication

Patent Publication 1: Specification of U.S. Pat. No. 6,165,678
Patent Publication 2: Specification of U.S. Pat. No. 6,177,228
Patent Publication 3: Japanese Patent Application Publication No. 2007-525696
Patent Publication 4: Japanese Patent Application Publication No. 2005-281301

### Non-Patent Publication

Non-Patent Publication 1: Zhurnal OrganicheskoiKhimii (Russia), (1995), 31 (3), p. 357-64

### SUMMARY OF THE INVENTION

In relation to the synthetic process as discussed in the above-mentioned production process (a), Patent Publication 3 sets forth a process using methylene chloride as a reaction solvent. However, methylene chloride is a deleterious substance so as not to be preferably used in large quantity on an industrial scale.

Additionally, the processes of Patent Publication 3 and Patent Publication 4 both are one in which a fluoroalkylsulfonic acid derivative is reacted with ethanolamine derivatives having a polymerizable double bond thereby causing sulfonylamidation. Particularly in the case of reacting a fluoroalkylsulfonic halide, there arises a problem of causing an addition reaction of halogen at the double bond moiety as a side reaction. In the case of using fluoroalkylsulfonic acid chloride, 1 to 6% of a chlorine adduct represented by the following formula is to be formed as a by-product.

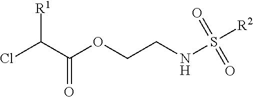

(In the formula, R¹ and R² represent the same to the above.)

A fluoroalkylsulfonylaminoethyl α-substituted-acrylate represented by the general formula [5] in particular is used as a raw material monomer for a resist material, so that contamination of such a by-product thereto is not preferable.

Moreover, when performing a further reaction on a compound having a polymerizable double bond, the double bond moiety may be cleaved by heat or light thereby possibly accelerating the polymerization. This is not preferable particularly in an industrial scale production, because heat or light conditions in some operations such as preparation, reaction, purification and the like sometimes cannot be controlled. It is thus preferable that an esterification step relating to the introduction of an acrylic acid moiety for obtaining a fluoroalkylsulfonic acid derivative is conducted in the final stage from the viewpoint of reducing impurities derived from the double bond.

On the other hand, with regard to the process (b), neither Patent Publication 1 nor Patent Publication 2 sets forth synthesis of the fluoroalkylsulfonylaminoethanol represented by the general formula [3], as discussed above. As the method therefor, a step of sulfonylamidating ethanolamine with a fluoroalkylsulfonic acid derivative will occur to those skilled in the art; however, formation of the target fluoroalkylsulfonylaminoethanol represented by the general formula [3] is not confirmed in a reaction between aminoethanol and a fluorosulfonic acid fluoride discussed in Example 4 of Patent Publication 4, so that it has been indicated that an industrial scale production with the process (b) has problems.

Thus a process for obtaining the fluoroalkylsulfonylaminoethanol represented by the general formula [3] with good yields has not yet been established under the current circumstances and therefore a process according to the process (b) has not yet been studied, which is the actuality.

An object of the present invention is to provide a production process for industrially efficiently producing the fluoroalkylsulfonylaminoethanol represented by the general formula [3]

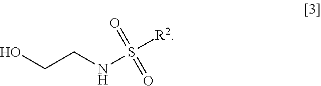

(In the formula R² represents the same to that discussed above.), as a production process alternative to the above-mentioned conventional process (a), and to provide a production process of efficiently synthesizing the fluoroalkylsulfonylaminoethyl α-substituted-acrylate represented by the general formula [5].

In view of such problems involved in conventional techniques, the present inventors have eagerly made studies in order to establish a process for producing a fluoroalkylsulfonylaminoethanol which process is suitable for an industrial scale production. As a result of this, they have found that sulfonylamidation between aminoethanol and a fluoroalkylsulfonic anhydride proceeds by reacting them under specified conditions thereby obtaining the target fluoroalkylsulfonylaminoethanol and that it is possible to obtain the target fluoroalkylsulfonylaminoethyl α-substituted-acrylate with higher purities, higher yields and higher operating efficiency than in conventional techniques (the above-mentioned process (a)) by esterifying the fluoroalkylsulfonylaminoethanol with an α-substituted acrylate derivative, with which the present invention is accomplished.

More specifically, the present invention involves the following inventions “Invention 1” to “Invention 8” and provides a process for producing a fluoroalkylsulfonylaminoethanol and a process for producing a fluoroalkylsulfonylaminoethyl α-substituted-acrylate.

[Invention 1]

A process for producing a fluoroalkylsulfonylaminoethanol represented by the general formula [3]

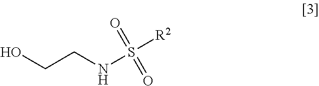

(In the formula, R² means the same to the above.), characterized by reacting aminoethanol represented by the general formula [1]

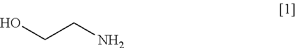

with a fluoroalkylsulfonic anhydride represented by the general formula [2]

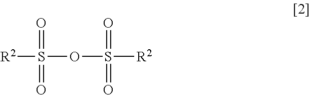

(In the general formula [2], R² represents a fluoroalkyl group having 1 to 6 carbon atoms.)

[Invention 2]

A production process as discussed in Invention 1, characterized in that the reaction is conducted in the absence of a base, at a composition ratio of 0.2 to 0.6 mol of the fluoroalkylsulfonic anhydride to 1 mol of the aminoethanol.

[Invention 3]

A production process as discussed in Invention 1, characterized in that the reaction is conducted in the presence of a base.

[Invention 4]

A production process as discussed in Invention 3, characterized in that the base is at least one kind of base selected from the group consisting of aminoethanol, trimethylamine, triethylamine, N,N-diethylmethylamine, tripropylamine, tributylamine, pyridine, 2,6-dimethylpyridine, dimethylaminopyridine, sodium carbonate, potassium carbonate, sodium hydroxide and potassium hydroxide.

[Invention 5]

A production process as discussed in any one of Invention 1 to Invention 4, characterized in that the reaction is conducted in the presence of a solvent.

[Invention 6]

A production process as discussed in Invention 5, characterized in that the solvent is at least one kind selected from the group consisting of acetonitrile, ethyl acetate, isopropyl alcohol, benzonitrile, dimethylimidazolidinone, dimethyl sulfoxide, diethyl ether, diisopropyl ether, dibutyl ether, tetrahydrofuran 1,1,2,2,3,3,4-heptafluorocyclopentane, trifluoromethyl benzene, 1,3-bis(trifluoromethyl)benzene and 1,4-bis(trifluoromethyl)benzene.

[Invention 7]

A production process as discussed in any one of Invention 1 to Invention 6, characterized in that the reaction is conducted at −50 to 50° C.

[Invention 8]

A process for producing a fluoroalkylsulfonylaminoethyl α-substituted-acrylate represented by the general formula [5]

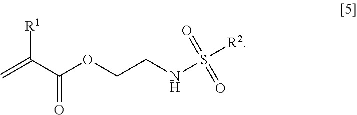

(In the formula, R¹ represents a hydrogen atom, methyl group, ethyl group, n-propyl group, isopropyl group, n-butyl group, sec-butyl group, tert-butyl group, fluoromethyl group, difluoromethyl group, trifluoromethyl group or perfluoroethyl group, and R² represents a fluoroalkyl group having 1 to 6 carbon atoms.), characterized in that a fluoroalkylsulfonylaminoethanol obtained by the process as discussed in any one of Invention 1 to Invention 7 and represented by the general formula [3] is reacted with an α-substituted acrylic acid derivative represented by the general formula [4]

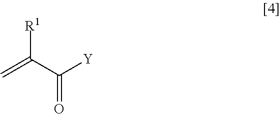

(In the formula, R¹ represents a hydrogen atom, methyl group, ethyl group, n-propyl group, isopropyl group, n-butyl group, sec-butyl group, tert-butyl group, fluoromethyl group, difluoromethyl group, trifluoromethyl group or perfluoroethyl group. Y represents any of a fluorine atom, chlorine atom, bromine atom and a group having a structure as shown in the following [4a].)

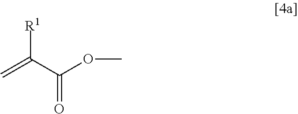

### DETAILED DESCRIPTION

According to the present invention, it is allowed to efficiently produce a fluoroalkylsulfonylaminoethanol (the raw material for a fluoroalkylsulfonylaminoethyl α-substituted-acrylate useful as a resist monomer) by an industrial process. Furthermore, it is possible to efficiently convert the fluoroalkylsulfonylaminoethanol into the fluoroalkylsulfonylaminoethyl α-substituted-acrylate with good operating efficiency, by providing the fluoroalkylsulfonylaminoethanol to an esterification reaction with an α-substituted acrylate.

Hereinafter, the present invention will be discussed in detail. The present invention relates to a process for producing a first target fluoroalkylsulfonylaminoethanol through a first step (a sulfonylamidation step), and the final target compound fluoroalkylsulfonylaminoethyl α-substituted-acrylate through a subsequent second step (an esterification step). The combination of the first step and the second step allows producing the final target compound fluoroalkylsulfonylaminoethyl α-substituted-acrylate (the general formula [5]) with higher yields and higher operating efficiency than in the conventional techniques. Moreover, the esterification step in the production process according to the present invention is performed at the final stage, with which formation of by-products including chlorine adduct and the like can be inhibited.

The reaction steps according to the present invention may be conducted by using a batch-type reactor. Conditions therefor will be discussed below, which does not limit modifications of the reaction conditions to those conceivable for those skilled in the art, even if concerning any reactor.

The first step (the sulfonylamidation step) is a step for sulfonylamidating aminoethanol represented by the general formula [1] with a fluoroalkylsulfonic anhydride represented by the general formula [2] thereby synthesizing a primary target compound of the present invention, i.e., a fluoroalkylsulfonylaminoethanol represented by the general formula [3]. A scheme of the present invention will be shown below.

First Step (Sulfonylamidation Step)

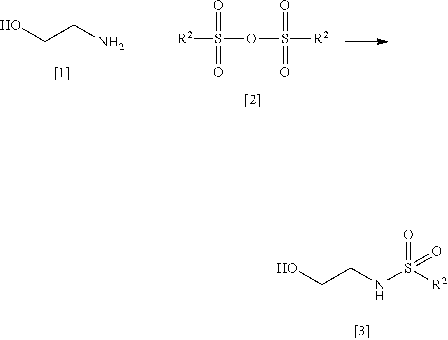

(In the Formula, R² represents the same to that discussed above.)

Aminoethanol used as a raw material for the sulfonylamidation step, represented by the general formula [1], may be a commercially available one or may be obtained by neutralizing a commercially available hydrochloride thereof with a base.

Meanwhile, in a fluoroalkylsulfonic anhydride (another raw material) represented by the general formula [2], R² represents a fluoroalkyl group having 1 to 6 carbon atoms. Examples of the fluoroalkyl group include a fluoromethyl group, difluoromethyl group, trifluoromethyl group, perfluoroethyl group, n-perfluoropropyl group, n-perfluorobutyl group and the like. In view of the utility of the product, the preferable are the perfluoroalkyl groups such as trifluoromethyl group, perfluoroethyl group, n-perfluoropropyl group, n-perfluorobutyl group and the like, and the more preferable is the trifluoromethyl group.

As discussed above, aminoethanol has two sites, i.e., an amino group and hydroxyl group. Hence the case where a fluoroalkylsulfonyl group is bound to each of them may raise the possibility of forming a by-product at a subsequent reaction, which is not preferable because the yield is to be reduced. Under normal circumstances, a sulfonylamidation reaction at an amine moiety due to an acid anhydride is to proceed antecedently, so that the target fluorosulfonylaminoethanol is to be produced with antecedency. In these circumstances, the important point on the reaction is how to inhibit a subsequent esterification reaction of hydroxyl group caused between the hydroxyl group and a fluoroalkylsulfonyl compound (an acid anhydride or acid).

In the first step (the sulfonylamidation step), a sulfonylating agent to be used is anhydride. Therefore, with proceedings of the sulfonylamidation reaction, 1 molecule of fluorosulfonic acid per 1 molecule of fluorosulfonic anhydride is produced. In order to prevent contact between such a free acid and the hydroxyl group moiety of the produced fluorosulfonylaminoethanol, there may be given a circumstance where the free fluorosulfonic acid is scavenged promptly.

Though bases including triethylamine and the like are effectively act as such a scavenger, the reaction between aminoethanol and a fluoroalkylsulfonic anhydride in the first step (the sulfonylamidation step) of the production process according to the present invention is to proceed in the presence or the absence of the bases. This is because the raw material aminoethanol is basic in itself. Hereinafter, a preferable embodiment of the ratio between aminoethanol and a fluoroalkylsulfonic anhydride, relating to each of the presence and the absence of the bases, will be discussed.

To begin with, a case of performing the first step (the sulfonylamidation step) in the presence of base will be discussed. In the reaction, base plays a role in trapping acids produced by the reaction. At least one kind selected from the group consisting of trimethylamine, triethylamine, N,N-diethylmethylamine, tripropylamine, tributylamine, pyridine, 2,6-dimethylpyridine, dimethylaminopyridine, sodium carbonate, potassium carbonate, sodium hydroxide and potassium hydroxide is preferably used as base. Of these, triethylamine is particularly preferable.

The amount of base to be used is 0.2 to 15.0 mol, preferably 0.5 to 10.0 mol, more preferably 1.0 to 3.0 mol relative to 1.0 mol of aminoethanol serving as a substrate. An amount of base less than 0.2 mol relative to the substrate aminoethanol decreases both the selectivity in the reaction and the yield of the target compound. Meanwhile, an amount of base exceeding 15.0 mol increases the amount of base which is not participate in the reaction so as not to be economically preferable. When an inexpensive one such as triethylamine is used as a solvent, an amount exceeding 15.0 mol is not problematic.

In the case of performing the reaction in the presence of base, the amount of a fluoroalkylsulfonic anhydride to be used is 0.2 to 2.0 mol, preferably 0.5 to 1.5 mol, more preferably 0.9 to 1.2 mol relative to 1.0 mol of aminoethanol. An amount of a fluoroalkylsulfonic anhydride less than 0.2 mol relative to 1.0 mol of aminoethanol decreases both the selectivity in the reaction and the yield of the target compound. Meanwhile, an amount of a fluoroalkylsulfonic anhydride exceeding 2.0 mol increases the amount of a fluoroalkylsulfonic anhydride which is not participate in the reaction thereby causing time and effort to dispose of it, which is therefore not economically preferable. In the case of performing the reaction in the presence of base as discussed above, the amount of a fluoroalkylsulfonic anhydride is preferably 0.9 to 1.2 mol relative to 1.0 mol of aminoethanol, and it is particularly preferable that the mole ratio between them is close to 1:1

When the reaction is conducted without base, aminoethanol is considered not only to serve as the raw material but also to act as the above-mentioned acid scavenger. Therefore, the mixture ratio of the raw materials preferably has such a composition as the amount of a fluoroalkylsulfonic anhydride is equal to or less than that of aminoethanol, and particularly preferably 0.2 to 0.6 mol relative to 1.0 mol of aminoethanol. When the amount of a fluoroalkylsulfonic anhydride relative to 1.0 mol of aminoethanol is smaller than 0.2 mol, the reaction efficiency is reduced. Meanwhile, that exceeding 0.6 mol not only decreases the acid-scavenging efficiency but also increases a fluoroalkylsulfonic anhydride which is not participate in the reaction thereby causing time and effort to dispose of it, which is therefore not economically preferable. Furthermore, it is preferable that the mole ratio therebetween is close to 1:0.5, the mole ratio allowing half the aminoethanol to scavenge free sulfonic acids. Such a condition makes it possible to achieve purification with not lower than 80% yield relative to the fluoroalkylsulfonic anhydride and not lower than 99% purity, which is a fact worthy to discuss (see Example 5).

Factors inhibiting esterification of a hydroxyl group of ethanolamine due to a fluorosulfonic anhydride include control of the reaction temperature. By carrying out the reaction under such conditions as to antecedently develop the sulfonylamidation and not to cause sulfonylesterification as much as possible, the formation of the by-products is able to be suppressed. The reaction temperature is within a range of from −50 to 50° C., preferably from −30 to 40° C., more preferably 0 to 30° C. If the reaction temperature is lower than −50° C., the reaction rate is too slow to provide a practical production process. Meanwhile, a reaction temperature exceeding 50° C. forms the by-products and therefore not preferable.

According to the present invention, the reaction proceeds with the addition of a fluoroalkylsulfonic anhydride to the raw material aminoethanol, in which a solvent may be used for improving the operating efficiency. Particularly in the case of conducting the reaction in the presence of base, fluoroalkylsulfonic acid salts of the base are formed as by-products, so that the solvent is preferably one capable of solving these by-products. A usable solvent is at least one kind of compound selected from the group consisting of a nitrile-based solvent such as acetonitrile, benzonitrile and the like, a sulfoxide-based solvent such as dimethyl sulfoxide and the like, an ether-based solvent such as diethyl ether, diisopropyl ether, dibutyl ether, tetrahydrofuran and the like, an alcohol-based solvent such as isopropyl alcohol and the like, a basic solvent such as triethylamine, pyridine and the like, a halogenated solvent such as methylene chloride, chloroform, carbon tetrachloride and the like, an aromatic hydrocarbon-based solvent such as benzene, xylene and the like, pentane, hexane and the like. Furthermore, in the present invention, a fluorine-based solvent may be used. The preferably usable thereof are 1,1,2,2,3,3,4-heptafluorocyclopentane, trifluoromethyl benzene, 1,3-bis(trifluoromethyl)benzene, 1,4-bis(trifluoromethyl)benzene and the like. The use of the fluorinated solvent is preferable because the solubility of the raw material and the product is thereby improved.

Additionally, the reaction time is not particularly limited and satisfactorily within a range of from 0.1 to 72 hours. Since the reaction time is changed according to the substrate and the reaction conditions, it is preferable to determine a temporal point at which a fluoroalkylsulfonic anhydride has been generally consumed as the endpoint of the reaction, by tracing the state of proceedings of the reaction in the use of an analytical device such as gas chromatography, liquid chromatography, NMR and the like.

Of these solvents, the nitrile-based solvent such as acetonitrile, benzonitrile and the like, the ether-based solvent such as diethyl ether, diisopropyl ether, dibutyl ether, tetrahydrofuran and the like, the basic solvent such as triethylamine, pyridine and the like, ethyl acetate, the fluorine-based solvent such as 1,1,2,2,3,3,4-heptafluorocyclopentane, trifluoromethyl benzene, 1,3-bis(trifluoromethyl)benzene, 1,4-bis(trifluoromethyl)benzene and the like are preferable.

The above-mentioned solvents may be used singly or as a mixture solvent combining two or more kinds thereof. For example, to mix at least one kind of the nitrile-based solvent including acetonitrile, benzonitrile and the like with at least one kind of the ether-based solvent including diethyl ether, diisopropyl ether, dibutyl ether, tetrahydrofuran and the like is superior, on an industrial scale, in further improving the solubility of the raw material and the product and allowing greatly shortening the reaction time, so as to be regarded as one preferable embodiment.

The amount of the solvent used in the present reaction is 0.5 to 10 g, preferably 1 to 5 g, more preferably 1 to 3 g relative to 1 g of aminoethanol. If the amount of the solvent is less than 0.5 g relative to 1 g of aminoethanol, the concentration of the by-products including the fluoroalkylsulfonic acid salts of the base precipitated during the reaction becomes too high so as to decrease the operating efficiency. An amount of the solvent exceeding 10 g is not economically preferable from the viewpoint of productivity.

A reactor with which the present reaction is conducted is preferably one formed of a tetrafluoroethylene resin, chlorotrifluoroethylene resin, vinylidene fluoride resin, PFA resin, glass or the like and subjected to lining at its inner section, or one formed of glass container.

An example of preferable embodiments of the present invention will be discussed in detail; however, the example does not limit the process for achieving the present invention.

A reactor resistant to the reaction conditions is charged with a base, a solvent and a raw material aminoethanol, followed by providing cooling for the raw material mixture by using a refrigerant, while stirring. After the temperature of the mixture is stabilized, a certain amount of a fluoroalkylsulfonic anhydride is added to the reaction mixture. Concerning the addition, it is possible to introduce the acid anhydride into a system bit by bit by successively or continuously conducting the addition. With this, free acids are not to be localized around a hydroxyl group of aminoethanol and therefore promptly trapped, even if generated, which is considered to suppress formation of the by-products. Additionally, it is preferable also because the reaction becomes consistent thereby allowing the reaction temperature to be readily controlled. Furthermore, it is preferable to confirm the termination of the reaction by monitoring consumption of the raw material, for example, by sampling.

A fluoroalkylsulfonylaminoethanol represented by the general formula [3] and produced by the process according to the present invention may be purified by applying a conventionally known process. However, the fluoroalkylsulfonylaminoethanol can be obtained at high purity in such a manner as to: rinse a reaction liquid with the addition of an acidic aqueous solution such as a hydrochloric acid aqueous solution, a sulfuric acid aqueous solution and the like, a basic aqueous solution containing sodium hydroxide, potassium hydroxide, sodium carbonate or the like, or an aqueous solution containing a salt to the reaction system; perform extraction with an organic solvent such as diisopropyl ether or the like; and then conduct purification such as distillation on a crude organic substance obtained with high purity.

A subsequent second step (an esterification step) is a step for esterifying the thus obtained fluoroalkylsulfonylaminoethanol (the general formula [3]) with an α-substituted acrylate (the general formula [4]) thereby producing the final target compound fluoroalkylsulfonylaminoethyl α-substituted-acrylate (the general formula [5]).

Second Step (Esterification Step)

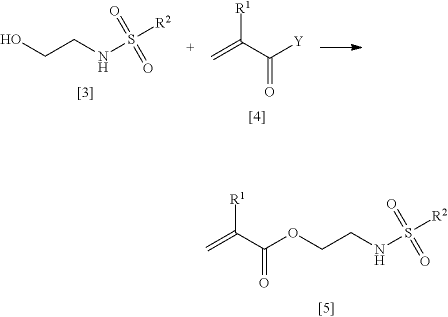

(In the formula of the above scheme, R¹ represents a hydrogen atom, methyl group, ethyl group, n-propyl group, isopropyl group, n-butyl group, sec-butyl group, tert-butyl group, fluoromethyl group, difluoromethyl group, trifluoromethyl group or perfluoroethyl group. Additionally, Y represents any of a fluorine atom, chlorine atom, bromine atom and a group having a structure as shown in the following [4a]. R² means the same to that discussed above.)

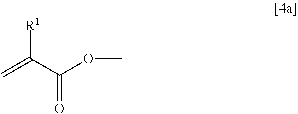

A fluoroalkylsulfonylaminoethyl α-substituted-acrylate represented by the general formula [5] is useful as a resist monomer. When used as the resist monomer, it is particularly preferable that R² is trifluoromethyl group in view of the utility. R¹ is preferably a hydrogen atom, methyl group, ethyl group, n-propyl group or isopropyl group, and particularly preferably methyl group. Of these, it is particularly preferable that R¹ is methyl group and R² is trifluoromethyl group.

Incidentally, an α-substituted acrylic acid derivative used in the esterification step as a reaction substrate can be commercially available as a reagent for synthesis.

The amount of an α-substituted acrylic acid derivative used in the esterification step is 0.8 to 3.0 mol, preferably 0.9 to 2.0, more preferably 0.9 to 1.5 mol relative to 1.0 mol of a fluoroalkylsulfonylaminoethanol. When the amount of an α-substituted acrylic acid derivative is smaller than 0.8 mol relative to 1.0 mol of a fluoroalkylsulfonylaminoethanol, the yield of the target compound is reduced. When it exceeds 3.0 mol, the amount of an α-substituted acrylic acid derivative which is not participate in the reaction is increased. This is not economically preferable in view of time and effort to dispose of it.

In the esterification step of the present invention, it is possible to use a solvent. There is no particular limit to usable solvents, but at least one kind of compound selected from aromatic hydrocarbon-based solvents such as benzene, toluene, xylene and the like, halogenated solvents such as methylene chloride, chloroform, carbon tetrachloride and the like, ether-based solvents such as diethyl ether, diisopropyl ether, dibutyl ether, tetrahydrofuran and the like, pentane, hexane and heptane is preferable. Of these, the aromatic hydrocarbon-based solvents such as benzene, toluene, xylene and the like, the halogenated solvents such as methylene chloride, chloroform, carbon tetrachloride and the like are particularly preferable.

The amount of the solvent used in the present reaction is normally 0.1 to 100 g, preferably 0.1 to 20 g, more preferably 2 to 10 g relative to 1 g of a fluoroalkylsulfonylaminoethanol. That exceeding 100 g is not preferable from the viewpoint of productivity.

The reaction temperature at which the reaction of the second step (the esterification step) is conducted is 20 to 200° C., preferably 40 to 150° C., more preferably 30 to 60° C. If the reaction temperature is lower than 20° C., the reaction rate is too slow to provide a practical production process. Meanwhile, a reaction temperature exceeding 200° C. makes a fluoroalkylsulfonylaminoethyl α-substituted-acrylate (the product) readily decomposable and therefore not preferable.

The esterification step of the present invention may be conducted by causing a reaction between a fluoroalkylsulfonylaminoethanol represented by the general formula [3] and an α-substituted acrylic acid derivative (an α-substituted acrylic anhydride or α-substituted acrylic halide) represented by the general formula [4] in the presence of a base.

A preferably usable base is at least one kind selected from the group consisting of trimethylamine, triethylamine, N,N-diethylmethylamine, tripropylamine, tributylamine, pyridine, 2,6-dimethylpyridine, dimethylaminopyridine, sodium carbonate, potassium carbonate, sodium hydroxide and potassium hydroxide. Of these, pyridine, 2,6-dimethylpyridine, triethylamine, sodium hydroxide are particularly preferable.

The amount of the base used in the present step is 0.2 to 2 mol, preferably 0.5 to 1.5 mol, more preferably 0.9 to 1.2 mol relative to 1 mol of a fluorosulfonylaminoethanol represented by the general formula [3]. With an amount of the base less than 0.2 mol relative to 1 mol of the fluorosulfonylaminoethanol represented by the general formula [3], both the selectivity in the reaction and the yield of the target compound are decreased. Meanwhile, an amount of the base exceeding 2 mol increases the amount of base which is not participate in the reaction and therefore not economically preferable.

Esterification can be achieved in the presence of the base as discussed above, but the reaction may be conducted with the addition of an acid serving as an additive in the case where an α-substituted acrylic acid derivative represented by the general formula [4] is an α-substituted acrylic anhydride in particular.

Conceivable examples of the additive (an acid) to be used are proton acids and Lewis acids in general, and organic sulfonic acids such as methanesulfonic acid, ethanesulfonic acid, p-toluenesulfonic acid, benzenesulfonic acid, trifluoromethanesulfonic acid and the like, but not particularly limited to these.

The amount of the additive to be used in the present reaction is 0.01 to 2 mol, preferably 0.02 to 1.8 mol, more preferably 0.05 to 0.5 mol relative to 1 mol of a fluorosulfonylaminoethanol represented by the general formula [3].

When the amount of the additive relative to 1 mol of a fluorosulfonylaminoethanol serving as a substrate and represented by the general formula [3] is lower than 0.01 mol, both the conversion ratio of the reaction and the yield of the target compound is decreased. Meanwhile, when it exceeds 2 mol, the amount of additive which is not participate in the reaction is increased and therefore not economically preferable.

For the purpose of preventing polymerization of the raw material (an α-substituted acrylic anhydride or α-substituted acrylic halide) or the product (a fluoroalkylsulfonylaminoethyl α-substituted-acrylate), the reaction of the present step may be conducted in the coexistence of a polymerization inhibitor.

The polymerization inhibitor that can be used is at least one kind of compound selected from hydroquinone, methoquinone, 2,5-di-t-butylhydroquinone, 1,2,4-trihydroxybenzene, 2,5-bistetramethylbutylhydroquinone, leukoquinizarin, nonflex F, nonflex H, nonflex DCD, nonflex MBP, ozonone 35, phenothiazine, 2-methoxyphenothiazine, tetraethylthiuram disulfide, 1,1-diphenyl-2-picrylhydrazyl, 1,1-diphenyl-1-picrylhydrazine, Q-1300 and Q-1301. The above-mentioned polymerization inhibitors are commercially available and therefore ready to get. The amount of the polymerization inhibitor used in the present step is 0.00001 to 0.1 mol, preferably 0.00001 to 0.05 mol, more preferably 0.0001 to 0.01 mol relative to the raw material fluorosulfonylaminoethanol represented by the general formula [3].

The amount of the polymerization inhibitor, exceeding 0.1 mol relative to the raw material fluorosulfonylaminoethanol represented by the general formula [3], does not bring about a dramatic difference in a polymerization-preventing ability. Therefore, this is not economically preferable.

A reactor with which the esterification reaction is performed is preferably one formed of a tetrafluoroethylene resin, chlorotrifluoroethylene resin, vinylidene fluoride resin, PFA resin, glass or the like and subjected to lining at its inner section, one formed of glass container, or one formed of stainless steel.

Though a process for achieving the present reaction is not limited, one example of preferable embodiments thereof will be discussed in detail.

A solvent and a fluoroalkylsulfonylaminoethanol are weighed and charged into a reactor resistant to the reaction conditions, followed by adjusting the temperature while stirring. After the temperature of the mixture is stabilized, a certain amount of an α-substituted acrylic acid derivative is added thereto. It is preferable to confirm the termination of the reaction by monitoring consumption of the raw material, for example, by sampling. Though a process for carrying out this reaction is not limited, one example of preferable embodiments thereof will be discussed in detail.

A reactor resistant to the reaction conditions is charged with an acid, base or metal catalyst, a solvent, the raw material fluorosulfonylaminoethanol represented by the general formula [3], an α-substituted acrylic acid derivative (an α-substituted acrylic halide or α-substituted acrylic anhydride) and a polymerization inhibitor, followed by adjusting the temperature while stirring, thereby developing a reaction. It is preferable to confirm the termination of the reaction by monitoring consumption of the raw material, for example, by sampling and to cool down a reaction liquid.

A fluoroalkylsulfonylaminoethyl α-substituted-acrylate produced by the present invention and represented by the general formula [5] may be purified by applying a conventionally known method such as rinsing, condensation, distillation, extraction, recrystallization, filteration, column chromatography and the like. Two or more kinds of these methods may be employed in combination.

### EXAMPLES

Referring now to Examples, the present invention will be specifically explained hereinafter; however, the present invention is not limited to these examples.

### Example 1

A 200 mL four-neck flask having a thermometer and a reflux condenser was charged with 30 g of acetonitrile and 30.5 g (0.50 mol) of ethanolamine, followed by cooling it to −15° C. while stirring. After 20 minutes of stirring, 74.6 g (0.26 mol) of trifluoromethanesulfonic anhydride was added dropwise over 90 minutes. After the dropwise addition was terminated, the temperature was increased to about 20° C. with stirring. Stirring was further continued for 3 hours. After stirring is terminated, 109 g of diisopropyl ether and 35 g of 10% hydrochloric acid aqueous solution were added thereto and then stirred for a while, followed by performing liquid separation. An organic layer was rinsed with 35 g of saturated sodium hydrogencarbonate aqueous solution 3 times, and then rinsed with 35 g of saturated brine. All of a water layer was brought together, followed by extracting it with 145 g of diisopropyl ether. The combined organic layer was The combined organic layer dried with magnesium sulfate, followed by distilling the solvent off. As a result of this, 38.8 g of a clear colorless liquid, i.e., crude 2-(trifluoromethyl)sulfonylaminoethanol was obtained. The crude yield was 80.4% and the GC purity was 86.1%. By conducting flash distillation (0.3 kPa/105-106° C.) thereon, 26.1 g of 2-(trifluoromethyl)sulfonylaminoethanol at 97.0% GC purity was obtained. The yield thereof was 54.1%.

¹H NMR (Solvent: CDCl₃, Reference material; TMS); δ 1.83 (s, 1H), 3.45 (t, J=5.2 Hz, 2H), 3.83 (t, J=5.2 Hz, 2H), 5.67 (s, 1H)

¹⁹F NMR (Solvent: CDCl₃, Reference material: CFCl₃); δ −77.94 (s, 1F)

### Example 2

A 30 mL flask was charged with 6.4 g of acetonitrile and 1.0 g (16.4 mmol) of ethanolamine, followed by cooling it to 0° C. while stirring. Then, 4.9 g (17.2 mmol) of trifluoromethanesulfonic anhydride was added dropwise thereto over 5 minutes. After the dropwise addition was terminated, stirring was further continued for 3.5 hours at 0° C. and then 10 g of 10% hydrochloric acid aqueous solution was added thereto, followed by extracting a water layer with 36 g of diisopropyl ether. An organic layer was rinsed with 10 g of saturated sodium hydrogencarbonate aqueous solution 2 times and then rinsed with 10 g of saturated brine, followed by drying it with magnesium sulfate. As a result of distilling the solvent off, 1.2 g of a brown liquid, i.e., a crude organic substance was obtained. The obtained crude organic substance was rinsed with 20 g of 10% sodium hydroxide aqueous solution upon the addition of 36 g of diisopropyl ether, followed by performing liquid separation. The water layer was made acidic with the addition of 10% hydrochloric acid aqueous solution and then extracted with 36 g of diisopropyl ether. The organic layer was rinsed with 10 g of saturated brine and then dried with magnesium sulfate. As a result of distilling the solvent off, formation of a brown liquid, i.e., crude 2-[(trifluoromethyl)sulfonyl]aminoethanol was confirmed by ¹H NMR.

### Example 3

A 30 mL flask was charged with 6.4 g of acetonitrile and 1.0 g (16.4 mmol) of ethanolamine, followed by cooling it to 0° C. while stirring. After 1.7 g (17.2 mmol) of triethylamine was added thereto and then stirred for 15 minutes, 4.9 g (17.2 mmol) of trifluoromethanesulfonic anhydride was added dropwise thereto over 5 minutes. After the dropwise addition was terminated, stirring was further continued for 3.5 hours at 0° C. and then 10 g of 10% hydrochloric acid aqueous solution was added thereto, followed by extracting a water layer with 36 g of diisopropyl ether. An organic layer was rinsed with 10 g of saturated sodium hydrogencarbonate aqueous solution 2 times and then rinsed with 10 g of saturated brine, followed by drying it with magnesium sulfate. Then, the solvent was distilled off, with which a brown liquid, i.e., 1.5 g of crude 2-[(trifluoromethyl)sulfonyl]aminoethanol was obtained (46% crude yield).

### Example 4

A 3 L four-neck flask having a thermometer and a reflux condenser was charged with 150 g of acetonitrile, 750 g of diisopropyl ether and 300 g (4.91 mol) of ethanolamine, followed by cooling it to 0° C. while stirring. After 30 minutes of stirring, 693 g (2.45 mol) of trifluoromethanesulfonic anhydride was added dropwise over 3 hours. After the dropwise addition was terminated, the temperature was decreased to about 10° C. with stirring over 20 minutes. After stirring is terminated, 889 g of 10% hydrochloric acid aqueous solution was added thereto and then stirred for a while, followed by performing liquid separation. Upon conducting extraction on a water layer with 300 g of diisopropyl ether, an organic layer was brought together and rinsed with 300 g of water, followed by distilling the solvent off. As a result of this, 424 g of a light pink liquid, i.e., crude 2-(trifluoromethyl)sulfonylaminoethanol was obtained. By conducting flash distillation on the crude product, 306 g of 2-(trifluoromethyl)sulfonylaminoethanol was obtained. The yield thereof was 65%.

### Example 5

A 3 L four-neck flask having a thermometer and a reflux condenser was charged with 100 g of acetonitrile, 500 g of diisopropyl ether and 300 g (4.91 mol) of ethanolamine, followed by cooling it to 0° C. while stirring. After 30 minutes of stirring, 693 g (2.45 mol) of trifluoromethanesulfonic anhydride was added dropwise over 3 hours. After the dropwise addition was terminated, the temperature was decreased to about 10° C. with stirring over 20 minutes. After stirring is terminated, 613 g of 16% sodium hydroxide aqueous solution was added thereto and then stirred for a while. Then, 252 g of 35% hydrochloric acid aqueous solution was added thereto with stirring for a while, followed by liquid separation. Upon rinsing a water layer with 150 g of diisopropyl ether 2 times, an organic layer was brought together and the solvent was distilled off. As a result of this, 456 g of a light pink liquid, i.e., crude 2-(trifluoromethyl)sulfonylaminoethanol was obtained. By conducting flash distillation on the crude product, 380 g of 2-(trifluoromethyl)sulfonylaminoethanol was obtained. The yield was 80% and the purity was 99%.

### Example 6

A 500 mL four-neck flask having a thermometer and a reflux condenser was charged with 250 g (1.30 mol) of 2-[(trifluoromethyl)sulfonyl]aminoethanol and 12.5 g (0.13 mol) of methanesulfonic acid with stirring, and then stirred at about 35° C. Then, 200 g (1.30 mol) of methacrylic anhydride was added dropwise thereto over 45 minutes. Incidentally, the maximal internal temperature during the dropwise addition was 47.0° C. After the dropwise addition was terminated, stirring was further continued for 4 hours at about 35° C. After stirring is terminated, it was cooled to 25° C. and 500 g of diisopropyl ether was added thereto. Then, 777 g of 8% sodium hydroxide aqueous solution was added thereto over 20 minutes, followed by stirring for 40 minutes at 40° C. Upon conducting liquid separation, an organic layer was added to 109 g of 5% sodium hydrogencarbonate, followed by stirring at 40° C. for 30 minutes. Upon conducting liquid separation, it was rinsed at 40° C. 3 times with the addition of 250 g of water, followed by distilling the solvent off. As a result of this, 313 g of a light yellow crystal, i.e., a crude organic substance was obtained. The crude yield was 93%. On the crude product, recrystallization was carried out with 127 g of diisopropyl ether and 1015 g of heptane. As a result, 278 g of the target substance, i.e., 2-{[(trifluoromethyl)sulfonyl]amino}ethyl 2-methylacrylate was obtained. The yield was 82% and the GC purity was 99.9%.

1-8. (canceled)

9. A process for producing a fluoroalkylsulfonylaminoethanol represented by the general formula [3] 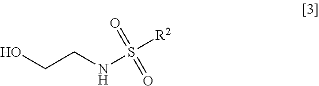
the process comprising the step of reacting aminoethanol represented by the general formula [1] 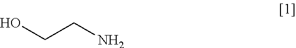
with a fluoroalkylsulfonic anhydride represented by the general formula [2] 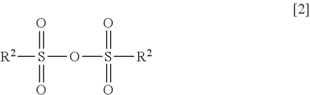
where R² represents a fluoroalkyl group having 1 to 6 carbon atoms.

10. A production process as claimed in claim 9, wherein the reaction is conducted in the absence of a base, at a composition ratio of 0.2 to 0.6 mol of the fluoroalkylsulfonic anhydride to 1 mol of the aminoethanol.

11. A production process as claimed in claim 9, wherein the reaction is conducted in the presence of a base.

12. A production process as claimed in claim 11, wherein the base is at least one kind of base selected from the group consisting of aminoethanol, trimethylamine, triethylamine, N,N-diethylmethylamine, tripropylamine, tributylamine, pyridine, 2,6-dimethylpyridine, dimethylaminopyridine, sodium carbonate, potassium carbonate, sodium hydroxide and potassium hydroxide.

13. A production process as claimed in claim 9, wherein the reaction is conducted in the presence of a solvent.

14. A production process as claimed in claim 13, wherein the solvent is at least one kind selected from the group consisting of acetonitrile, ethyl acetate, isopropyl alcohol, benzonitrile, dimethylimidazolidinone, dimethyl sulfoxide, diethyl ether, diisopropyl ether, dibutyl ether, tetrahydrofuran 1,1,2,2,3,3,4-heptafluorocyclopentane, trifluoromethyl benzene, 1,3-bis(trifluoromethyl)benzene and 1,4-bis(trifluoromethyl)benzene.

15. A production process as claimed in claim 9, wherein the reaction is conducted at −50 to 50° C.

16. A process for producing a fluoroalkylsulfonylaminoethyl α-substituted-acrylate represented by the general formula [5] 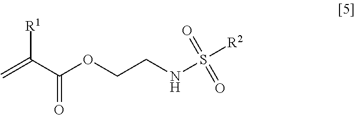
the process comprising the step of reacting a fluoroalkylsulfonylaminoethanol obtained by the process as claimed in claim 9 and represented by the general formula [3] with an α-substituted acrylic acid derivative represented by the general formula [4] 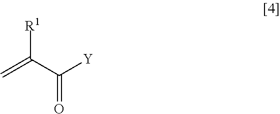
where R¹ represents a hydrogen atom, methyl group, ethyl group, n-propyl group, isopropyl group, n-butyl group, sec-butyl group, tert-butyl group, fluoromethyl group, difluoromethyl group, trifluoromethyl group or perfluoroethyl group, Y represents one of a fluorine atom, chlorine atom, bromine atom and a group having a structure as shown in the following [4a] 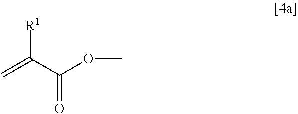
and R² represents a fluoroalkyl group having 1 to 6 carbon atoms.
